(19) United States
(12) Patent Application Publication
(10) Pub. No.: US 2014/0275170 A1
(43) Pub. Date: Sep. 18, 2014

(54) TASTE MASKING OF ANESTHETICS AND ANALGESICS

(71) Applicant: JSN I, LLC, Longwood, FL (US)

(72) Inventors: Jeremy Nehleber, Longwood, FL (US); Neil Ross, Weston, FL (US)

(73) Assignee: JSN I, LLC, Longwood, FL (US)

(21) Appl. No.: 14/211,000

(22) Filed: Mar. 14, 2014

Related U.S. Application Data
(60) Provisional application No. 61/784,351, filed on Mar. 14, 2013.

(51) Int. Cl. A61K 31/445; A61K 31/137; A61K 9/00; A61K 31/167

(52) U.S. Cl. CPC A61K 31/445; A61K 31/167; A61K 31/137; A61K 9/0056
USPC 514/330; 514/626

(57) ABSTRACT

The present invention improves the bad taste of injected anesthetics and analgesics by incorporating a flavoring or cooling agent such as menthol into the parenteral composition.

### PRIORITY CLAIM AND CROSS REFERENCE

This application claims priority to U.S. provisional patent application No. 61/784,351 filed on Mar. 14, 2013, the contents of which are expressly incorporated by reference. The contents of all cited references are expressly incorporated by reference.

### BACKGROUND

In the current practice of dentistry, injectable anesthetics are administered to provide comfort and a more desirable experience for the patient. Lidocaine, Carbocaine, Bupivacaine and other injectable anesthetics and analgesic drugs show a metallic/bitter aftertaste after injection. This taste can last up to 2 hours after the anesthetic dissipates. This taste is highly objectionable to all patients and in many it can cause upset stomach and sometimes nausea. Frequently, patients describe the unpleasant taste of injectable anesthetics as a factor contributing to the unpleasantness of the procedure. This experience may impact an individual from obtaining more timely treatments due to this unpleasantness. Historically, very little has been done to help mitigate this problem.

Bonaparte J P, Corsten M, Rourke R. Reducing the flavour of oral lidocaine: randomized controlled trial assessing the efficacy of mint-flavoured mouthwash. J Otolaryngeal Head Neck Surgery 2010 February; 39 (1): 96-101 tried an oral rinse using peppermint or spearmint mouth type washes to eliminate the taste with limited success. To date, the efforts to eliminate the bad taste of drugs injected into the oral cavity have been unsuccessful. A bad experience by a patient may prevent an individual from obtaining treatment due to the unpleasantness of the anesthetic.

While menthol has been used as an injectable anesthetic alone, the art does not show its use to mask the taste of other injectable agents. Water-soluble menthol β-hydroxypropyl cyclodextrin has been developed as a long-acting injection painkiller. There remains a need in the art for an anesthetic which does not have an objectional taste.

### BRIEF SUMMARY OF THE INVENTION

To improve the taste issue of injected anesthetics and analgesics, the use of a flavor agent in the parental solution should be employed. In a preferred embodiment the anesthetics are selected from lidocaine, mepivicaine, septocaine or bupivacaine in combination with a taste masking agent such as menthol in an acceptable solvent.

### DETAILED DESCRIPTION OF THE INVENTION

It has been surprisingly discovered that the bad tastes that a patient experiences from certain injectable compositions can be masked by adding a flavor masking agent to the injectable composition.

To improve the taste issue of anesthetics and analgesic injections, the use of a flavor agent in the parental solution should be employed. Suitable active agents include, but are not limited to, lidocaine (also called xylocaine or lignocaine), novocaine and procaine, articaine, septocaine, marcainemidazolam, ketamine, propofol and fentanyl (a long-acting anesthetic), bupivacaine, and mepivacaine (Carbocaine). In a preferred embodiment the anesthetics are selected from lidocaine, mepivacaine, septocaine or bupivacaine in combination with a taste masking agent such as menthol in an acceptable solvent. A combination of these may be used depending on the situation. Also, most agents come in two forms: with and without epinephrine. The flavor masking agents may cause a temporary loss of taste sensation to the taste buds.

Suitable flavoring agents include, but are not limited to, ascorbic acid, menthol, peppermint, spearmint, wintergreen, butterscotch, chocolate, cocoa, pineapple, orange, any citrus based flavor, any mint based flavor, any cooling agent or any natural flavor.

Cooling agents are known to provide a “cool” sensation or cooling effect on or following application to a body surface; and substances that, on or following topical application can ameliorate a hot sensation or a heating effect are called “cooling agents”. Any agent which, when incorporated in the injectable composition in a concentration sufficient to exert a cooling sensation or effect or to ameliorate a hot sensation or heating effect, is suitable for use a cooling agent in accordance with the present invention. The cooling agent may be an excipient, an active ingredient or pharmaceutical, therapeutic or cosmetic agent or a propellant or combinations of two or more agents. Non limiting examples of cooling agents, which are all incorporated herein by reference are provided in U.S. Pat. Nos. 3,419,543, 4,020,153, 4,032,661, 4,033,994, 4,034,109, 4,059,118, 4,060,091, 4,070,449, 4,070,496, 4,136,163, 4,150,052, 4, 4,153,679, 157,384, 4,178,459, 4,193,936, 4,226,988, 4,230,688, 4,296,255, 4,459,425, 5,266,592, 5,608,119, 5,725,865, 5,773,410, 5,843,466, 5,959,161, 6,214,788, 6,267,974, 6,303,817, 6,328,982, 6,359,168, 6,482,983, 6,592,884, 6,884,906 and 7,030,273 US Patent Application 20040067970, DE 2,202,535, DE 2,205,255, DE 2,503,555, DE 2,608,226, DE 4,226,043, GB 1,351,761, GB 1,351,762, GB 1,421,744, J P 2004059474 and WO 2005049553; and in Watson et al, J. Soc. Cosmet. Chem. 29, 185-200 (1978); and Ottinger et al in J. Agric. Food Chem., 49, 5383-5390 (2001).

In additional embodiment, the cooling agent is an isomer or a derivative of menthol. Additional examples of menthol derivatives are monomenthyl esters of di- and polycarboxylic acids. Some derivatives have been developed to be substantially without smell. Useful exemplary menthol derivatives are menthol ethylene glycol carbonate, which is now known as Frescolat® type MGC, enthol Propylene Glycol Carbonate (Frescolat® type MPC), menthyl lactate (Frescolat ML®) and Menthone Glycerin Acetal (Frescolat MGA®).Additional widely used menthol derivatives are 3-(/-Menthoxy)-1,2-propanediol, known as Cooling Agent 10; and the same structure with an additional methyl group in the glycerin part of the molecule: Menthoxy-coolants are additional cooling menthol derivatives Cubebol is an example of a coolant with a C—C bond in the 3d position of p-menthane.

Cooling agents, which are not menthol derivatives, are also suitable for use in accordance with the present invention. Examples of such cooling agents are provided below: Exemplary cooling agents which can be used in the invention include, but are not limited to, menthol, isopulegol, 3-(1-menthoxy)propane-1,2-diol, 3-(1-menthoxy)-2-methylpropane-1,2-diol, p-menthane-2,3-diol, p-menthane-3,8-diol, 6-isopropyl-9-methyl-1,4-dioxas-piro[4,5]decane-2-methanol, menthyl succinate and its alkaline earth metal salts, trimethylcyclohexanol, N-ethyl-2-isopropyl-5-methylcyclohexanecarb-oxamide, Japanese mint (Mentha arvensis) oil, peppermint oil, menthone, menthone glycerol ketal, menthyl lactate, 3-(1-menthoxy)ethan-1-ol, 3-(I-menthoxy)propan-1-ol, 3-(I-menthoxy)butan-1-ol, I-menthylacetic acid N-ethylamide, I-menthyl-4-hydroxypentanoate, I-menthyl-3-hydroxybutyrate, N,2,3-trimethyl-2-(1-methylethyl)-butanamide, and spearmint oil.

The solvents that can be used are any solvent capable of solubilizing the components preferred: PEG (Polyethylene Glycols), Chloroform, Ethanol, Carvone, Dimethylsulfoxide, Citral, Tauroglycolate, Eugenol, Sodium Lauryl Sulfate, Oleic acid, Tween, N Methyl 2 pyrrilidione, 2 pyrol, PVP (Polyvinylpyrrilidones) or any alcohol and water. In a one embodiment, the solvent is PEG in a range of 0.5-70% by weight with 5 to 25% by weight preferred, with 5-10% by weight most preferred.Plasticizers that can be used for the product are: Glycerol and esters, Glycol derivatives, phthalix acid esters, oleic acid esters, sugar alcohols, citric acid esters, tartaric acid esters, PEG (Polyethylene Glycols), PVP, Eugradit (methyl acrylates), Cellulose esters. Plastisizers can be utilized to improve water dispersion of the flavor agent and prevent crystallization in the water solution.

The dose of any local anesthetic administered varies with the anesthetic procedure, the area to be anesthetized, the vascularity of the tissues, the number of neuronal segments to be blocked, the depth of anesthesia and degree of muscle relaxation required, the duration of anesthesia desired, individual tolerance and the physical condition of the patient. The smallest dose and concentration required to produce the desired result should be administered. The rapid injection of a large volume of local anesthetic solution should be avoided and fractional doses should be used when feasible.

Currently marketed products for local anesthesia by injection for dentistry include, but are not limited to Mepivacaine 3% and Mepivicaine 2% with Levonordefrin sold under the brand Isocaine by Novocol; Carbocain 2% with Neo-Cobefrin sole by Cook-Waite; Lidocaine 2% with epinepherine 1:100,000 and Lidocaine 2% with epinepherine 1:50,000 sold by Superdent; Lidocaine 2% with epinepherine 1:100,000 sold by Patterson Dental Supply, Inc.; articain 4% and epinepherine 1:100,000 sold under the brand name Septocaine by Septodont; Mepivacaine 3% sold under the brand name Scandonest by Septodont; Lidocaine 2% with epinepherine 1:50,000 and Lidocaine 2% with epinepherine 1:100,000 sold under the brand name Xylocaine Dental by Dentsply Pharmaceutical; sold under the brand name Xylocaine Dental by Dentsply Pharmaceutica; the instructions for use for which are expressly incorporated by reference.

While the specific examples below are described in terms of dental formulations, one of skill in the art will readily understand they can be used anytime taste masking is required for an injectable composition.

### EXAMPLE 1

### Lidocaine

Table 1 shows an example of a lidocaine type product:

| | | mg/ serving | Concentrate | INGREDIENT | | |
|---|---|---|---|---|---|---|
| factor | mg/ unit | final dosage mg | content mg | DESCRIPTION | EXC | gm/BATCH |
| 0.97 | 10 | 10 | 10.3092784 | Lidocaine USP 97% | 1.00 | 20.6186 |
| 0.99 | 0.005 | 0.005 | 0.00505051 | Epinephrine USP 99% | 1.00 | 0.0101 |
| 1 | 1000 | 1000 | 1000 | water for injection | 1.00 | 2000.00 |
| | | | | USP, sterile | | |
| 1 | 1 | 1 | 1 | Ascorbic Acid | 1.00 | 2.00 |
| | | | | 98% Inj USP | | |
| 1 | 1 | 1 | 1 | Menthol EP | 1.00 | 2.00 |
| 1 | 0.5 | 0.5 | 0.5 | Sodium | 1.00 | 1.00 |
| | | | | Metabisulfate USP | | |
| | 1012.51 | 1012.505 | 1012.81433 | | | 2025.629 |

Process
1. Add lidocaine 100 cc to sterile 250 cc beaker
2. Weigh out 0.05 gm of L-Menthol EP
3. Weigh out 0.99 gm Sodium Chloride USP
4. Add Menthol and Sodium Chloride to 10 cc of Ethanol is a 50 ml beaker and stir the solution at 75 rpm for 5 minutes until dissolved.
5. Filter resulting solution using a
6. Add the ethanol solution to the lidocaine solution and stir the solution at 75 rpm for 5 minutes
7. Filter the solution using a 500 cc sterile filter bottle SVB 002 and vacuum pump VP001 to filter
8. Fill 20 cc of solution into 20 cc clear vials.
9. Seal and label vials.

### EXAMPLE 2

### Lidocaine with Epinephrine

| | | g/serving | Concentrate | INGREDIENT | | |
|---|---|---|---|---|---|---|
| factor | g/unit | final dosage | content g | DESCRIPTION | EXC | gm/BATCH |
| 1 | 50 | 50 | 50 | Lidocaine 2% Epinephrine | 1.00 | 50.0000 |
| | | | | 1:100000 sterile | | |
| | | | | solution | | |
| | | | | water for injection | | |
| | | | | USP, sterile | | |
| | | | | PEG 400 USP/EP | | |
| | | | | Sodium | | |
| | | | | Chloride USP | | |
| | | | | L-Menthol EP | | |
| 1 | 43 | 43 | 43 | | 1.00 | 43.0000 |
| 1 | 7 | 7 | 7 | | 1.00 | 7.0000 |
| 1 | 0.9 | 0.9 | 0.900 | | 1.00 | 0.9000 |
| 1 | 0.05 | 0.05 | 0.05 | | 1.00 | 0.0500 |
| | 100.95 | 100.95 | 100.95 | | | 100.950 |

1. Add lidocaine 50 g to sterile 250 cc beaker
2. Weigh out 0.05 gm of L-Menthol EP
3. Weigh out 0.900 gm Sodium Chloride USP
4. Add Menthol and Sodium Chloride to 10 cc of Ethanol is a 50 ml beaker and stir the solution at
75 rpm for 5 minutes until dissolved.
5. Filter resulting solution using
6. Add the ethanol solution to the lidocaine solution and stir the solution at 75 rpm for 5 minutes
7. Filter the solution using a 500 cc sterile filter bottle SVB 002 and vacuum pump VP001 to filter
8. Fill 20 cc of solution into 20 cc clear vials.
9. Seal and label vials.

### EXAMPLE 3

### Mepivicaine

Table 2 shows an example of a Mepivacaine type product:

| | | mg/serving | Concentrate | INGREDIENT | | |
|---|---|---|---|---|---|---|
| factor | mg/unit | final dosage mg | content mg | DESCRIPTION | EXC | gm/BATCH |
| 0.97 | 10 | 10 | 10.30927835 | Mepivacaine 98% | .00 | 2.0619 |
| | | | | Inj USP | | |
| 1 | 6.6 | 6.6 | 6.6 | Sodium Chloride USP | .00 | 1.3200 |
| 1 | 1000 | 1000 | 1000 | water for injection USP, sterile | .00 | 200.0000 |
| 1 | 0.5 | 0.5 | 0.5 | Menthol EP | .00 | 0.1000 |
| 1 | 0.3 | 0.3 | 0.3 | Potassium Chloride USP | .00 | 0.0600 |
| 1 | 0.33 | 0.33 | 0.33 | Calcium Chloride | .00 | 0.0660 |
| | | | | USP | | |
| 1 | 1 | 1 | 1 | Methylparaben USP | .00 | 0.2000 |
| | 1048.53 | 1048.53 | 1048.839278 | | waste | 209.768 |

Process
1. Add 2.0619 gm mepivicain 100 cc to sterile 250 cc beaker
2. Add 1.3200 gm Sodium Chloride
3. Add 200 ml water for injection USP to beaker and stir the solution at 75 rpm for 5 minutes
4. Filter the solution using a 500 cc sterile filter bottle SVB 002 and vacuum pump VP001 to filter
5. Add 0.1 gm of L-Menthol EP
6. Add 0.0600 gm of potassium chloride
7. Add 0.0660 gm of calcium chloride
8. Add 0.200 gm of Methylparaben USP
9. stir the solution at 75 rpm for 5 minutes
10. Filter the solution using a 500 cc sterile filter bottle SVB 002 and vacuum pump VP001 to filter
11. Fill 20 cc of solution into 20 cc clear vials.
12. Seal and label vials.

### EXAMPLE 4

### Bupivacaine

Table 3 shows an example of a bupivacaine type product:

| | | mg/serving | Concentrate | INGREDIENT | | |
|---|---|---|---|---|---|---|
| factor | mg/unit | final dosage mg | content mg | DESCRIPTION | EXC | gm/BATCH |
| 1 | 5 | 5 | 5 | bupivacaine HCl USP | .00 | 1.0000 |
| 0.99 | 0.005 | 0.005 | 0.005050505 | epinephrine USP 99% | .00 | 0.0010 |
| 1 | 1000 | 1000 | 1000 | water for injection | .00 | 200.0000 |
| | | | | USP, sterile | | |
| 1 | 0.5 | 0.5 | 0.5 | menthol EP | .00 | 0.1000 |
| 1 | 8.5 | 8.5 | 8.5 | sodium chloride USP | .00 | 1.7000 |
| 1 | 0.1 | 0.1 | 0.1 | edetate calcium | .00 | 0.0200 |
| | | | | disodium, anhydrous | | |
| 1 | 0.1 | 0.1 | 0.1 | sodium | 1.00 | 0.0200 |
| | | | | metabisulfate USP | | |
| | 1014.205 | 1014.205 | 1014.205051 | | waste | 202.841 |

Process
1. Add bupivacaine HCl 1 gm to sterile 250 cc beaker.
2. Add 0.0010 gm Epinephrine USP 99%.
3. Add 200 ml water for injection USP to beaker and stir the solution at 75 rpm for 5 minutes.
4. Filter the solution using a 500 cc sterile filter bottle SVB 002 and vacuum pump VP001 to filter.
5. Add 0.100 gm of L-Menthol EP.
6. Add 1.7 gm of sodium chloride.
7. Add 0.020 gm of edetate calcium disodium, anhydrous.
8. Add 0.020 gm of Sodium Metabisulfate USP.
9. stir the solution at 75 rpm for 5 minutes.
10. Filter the solution using a 500 cc sterile filter bottle SVB 002 and vacuum pump VP001 to filter.
11. Fill 20 cc of solution into 20 cc clear vials.
12. Seal and label vials.

### EXAMPLE 5

### Lidocaine with Epinephrine

Table 4 shows an example of a Lidocaine with Epinephrine type product:

| | | mg/serving | Concentrate | INGREDIENT | | |
|---|---|---|---|---|---|---|
| factor | mg/unit | final dosage mg | content mg | DESCRIPTION | EXC | gm/BATCH |
| 0.97 | 10 | 10 | 10.3092784 | lidocaine USP 97% | 1.00 | 10.3093 |
| 0.99 | 0.005 | 0.005 | 0.00505051 | epinephrine USP 99% | 1.00 | 0.0051 |
| 1 | 1000 | 1000 | 1000 | water for injection USP, | 1.00 | 1000.0000 |
| | | | | sterile | | |
| 1 | 30 | 30 | 30 | ethanol 100% USP | 1.00 | 30.0000 |
| 1 | 9.27 | 9.27 | 9.27 | sodium Chloride USP | 1.00 | 9.2700 |
| 1 | 0.3 | 0.3 | 0.3 | menthol EP | 1.00 | 0.3000 |
| 1 | 0.5 | 0.5 | 0.5 | sodium metabisulfate USP | 1.00 | 0.5000 |
| | 1050.08 | 1050.075 | 1050.38433 | | waste | 1050.384 |

1. Add Lidocain 10.3 gm to sterile 250 cc beaker.
2. Add 0.0051 gm Epinephrine USP 99%.
3. Add 1000 ml water for injection USP and 30 m1 ethanol 100% USP to beaker and stir the solution at 75 rpm for 5 minutes.
4. Filter the solution using a sterile filter bottle SVB 002 and vacuum pump VP001 to filter.
5. Add 0.300 gm of L-Menthol EP.
6. Add 9.27 gm of sodium chloride.
7. Add 0.5 gm of Sodium Metabisulfate USP.
8. stir the solution at 75 rpm for 5 minutes.
9. Filter the solution using a 500 cc sterile filter bottle SVB 002 and vacuum pump VP001 to filter.
10. Fill 20 cc of solution into 20 cc clear vials.
11. Seal and label vials.

### EXAMPLE 6

### Testing Protocol

Compositions of the present invention can be tested by using a split mouth design protocol administering, via mental block, the control, non-taste masked formulation on one side, while administering the present invention on the other side. We will then be able to assess the efficacy of the anesthetic by seeing if there is any change in duration. Taste will be tested utilizing a scale for taste. Either VAS (visual analogue scale) or another test.

### EXAMPLE 7

### Lidocaine Administration

Local administration in dental procedures

When used for local anesthesia in dental procedures the dosage of Dental lidocaine HCl) Injection depends on the physical status of the patient, the area of the oral cavity to be anesthetized, the vascularity of the oral tissues, and the technique of anesthesia. The least volume of solution that results in effective local anesthesia should be administered; time should be allowed between injections to observe the patient for manifestations of an adverse reaction. For specific techniques and procedures of a local anesthesia in the oral cavity, refer to standard textbooks.

For most routine dental procedures, 2% lidocaine with 1:100,000 epinephrine is preferred. However, when greater depth and a more pronounced hemostasis are required, a 1:50,000 epinephrine concentration should be used.

Dosage requirements should be determined on an individual basis. In oral infiltration and/or mandibular block, initial dosages of 1.0-5.0 mL of 2% lidocaine with epinephrine 1:50,000 or 1:100,000 are usually effective.

In children under 10 years of age it is rarely necessary to administer more than one-half cartridge (0.9-1.0 mL or 18-20 mg) of lidocaine per procedure to achieve local anesthesia for a procedure involving a single tooth. In maxillary infiltration, this amount will often suffice to the treatment of two or even three teeth. In the mandibular block, however, satisfactory anesthesia achieved with this amount of drug will allow treatment of the teeth in an entire quadrant. For children greater than 10 yrs of age and adults do not exceed 6.6 mg/kg

Systemic Administration

Single Direct Intravenous Injection (Bolus)

The usual dose is 50 to 100 mg of lidocaine hydrochloride (0.70 to 1.4 mg/kg; 0.32 to 0.63 mg/lb) administered intravenously under ECG monitoring. This dose may be administered at the rate of approximately 25 to 50 mg/min (0.35 to 0.70 mg/kg/min; 0.16 to 0.32 mg/lb/min). Sufficient time should be allowed to enable a slow circulation to carry the drug to the site of action. If the initial injection of 50 to 100 mg does not produce a desired response, a second dose may be injected after 5 minutes.

Continuous Intravenous Infusion

Following bolus administration, intravenous infusions of Xylocaine may be initiated at the rate of 1 to 4 mg/min of lidocaine hydrochloride (0.014 to 0.057 mg/kg/min; 0.006 to 0.026 mg/lb/min). The rate of intravenous infusions should be reassessed as soon as the patient's basic cardiac rhythm appears to be stable or at the earliest signs of toxicity. It should rarely be necessary to continue intravenous infusions for lidocaine for prolonged periods.

### EXAMPLE 8

### Mepivacaine Administration

The recommended single adult dose (or the total of a series of doses given in one procedure) of mepivacaine for unsedated, healthy, normal-sized individuals should not usually exceed 400 mg. The recommended dosage is based on requirements for the average adult and should be reduced for elderly or debilitated patients.

While maximum doses of 7 mg/kg (550 mg) have been administered without adverse effect, these are not recommended, except in exceptional circumstances and under no circumstances should the administration be repeated at intervals of less than 1.5; hours.

The total dose for any 24-hour period should not exceed 1,000 mg because of a slow accumulation of the anesthetic or its derivatives or slower than normal metabolic degradation or detoxification with repeat administration.

Pediatric patients tolerate the local anesthetic as well as adults. However, the pediatric dose should be carefully measured as a percentage of the total adult dose based on weight, and should not exceed 5 mg/kg to 6 mg/kg (2.5 mg/lb to 3 mg/lb) in pediatric patients, especially those weighing less than 30 lb. In pediatric patients under 3 years of age or weighing less than 30 lb concentrations less than 2% (e.g., 0.5% to 1.5%) should be employed.

TABLE 5
Mepivicaine Dosing

| | Concen- | Total Dose | | |
|---|---|---|---|---|
| Procedure | tration | mL | mg | Comments |
| Cervical, | 1% | 5-40 | 50-400 | Pudendal block: one |
| brachial, | 2% | 5-20 | 100-400 | half of total dose |
| intercostal, | | | | injected each side. |
| pudendal nerve | | | | |
| block | | | | |
| Transvaginal | 1% | up to 30 | up to 300 | One half of total dose |
| block (para- | | (both | (both | injected each side. |
| cervical plus | | sides) | sides) | |
| pudendal) | | | | |
| Paracervical | 1% | up to 20 | up to 200 | One half of total dose |
| block | | (both | (both | injected each side. |
| | | sides) | sides) | This is maximum |
| | | | | recommended dose |
| | | | | per 90 minute period |
| | | | | in obstetrical and |
| | | | | non-obstetrical |
| | | | | patients. Inject |
| | | | | slowly, 5 minutes |
| | | | | between sides. |
| Caudal and | 1% | 15-30 | 150-300 | Use only single-dose |
| epidural block | 1.5% | 10-25 | 150-375 | vials which do not |
| | 2% | 10-20 | 200-400 | contain a preservative. |
| Infiltration | 1% | up to 40 | up to 400 | An equivalent |
| | | | | amount of a 0.5% |
| | | | | solution (prepared by |
| | | | | diluting the 1% |
| | | | | solution with Sodium |
| | | | | Chloride Injection, |
| | | | | USP) may be used |
| | | | | for large areas. |
| Therapeutic | 1% | 1-5 | 10-50 | |
| block (pain | 2% | 1-5 | 20-100 | |
| management) | | | | |

### EXAMPLE 9

### Bupivacaine Administration

The 0.5% concentration with epinephrine is recommended for infiltration and block injection in the maxillary and mandibular area when a longer duration of local anesthetic action is desired, such as for oral surgical procedures generally associated with significant postoperative pain. The average dose of 1.8 mL (9 mg) per injection site will usually suffice; an occasional second dose of 1.8 mL (9 mg) may be used if necessary to produce adequate anesthesia after making allowance for 2 to 10 minutes onset time). The lowest effective dose should be employed and time should be allowed between injections; it is recommended that the total dose for all injection sites, spread out over a single dental sitting, should not ordinarily exceed 90 mg for a healthy adult patient (ten 1.8 mL injections of 0.5% BUPIVACAINE HCl with epinephrine). Injections should be made slowly and with frequent aspirations.

### EXAMPLE 10

### Clinical Testing

Formulations of the present invention were tested in patients to determine if they improved the taste over commercially available dental injectable anesthetics. Four patients were administered the compositions shown below together with a non-tasted masked commercially available product. Following administration subjects would then pick from a visual analog scale ranging from 1(best tasting)-10(worst tasting).

### A: Lidocaine Injectable: Ethanol Solvent 10%

A lidocaine preparation was prepared as in Example 5 having the following ingredient concentrations:

Lidocaine 1.8%

Epinephrine 1:1000000

Menthol 0.4%

4 subjects were first administered 20 IUs of a commercially available 2% lidocaine with 1:100000 epinephrine productorally with the composition placed directly onto dorsal of tongue and the subjects asked to evaluate taste. Table 6 shows the results:

TABLE 6

| Test Subject | Rating |
|---|---|
| 1 | 7 |
| 2 | 8 |
| 3 | 7 |
| 4 | 7 |

After subjects had water and a small white plain cracker, then 10 minutes later, all 4 subjects were administered 20 IUs of the composition of this example orally with the composition placed directly onto dorsal of tongue and the subjects asked to evaluate taste. Table 7 shows the taste results.

TABLE 7

| Test Subject | Rating |
|---|---|
| 1 | 3 |
| 2 | 3 |
| 3 | 3 |
| 4 | 2 |

After taste test was performed anesthesia was administered via a block of the mental nerve using a split mouth design in which on one side of the patients mouth 100 IUs of 2% lido w/1:100 k epi was administered via mental block. At the same time 100 IUs of the composition of this example was administered on the contralateral side.

All subjects had profound anesthesia on both sides, with the test sites having slightly more then the control. This could be due to the additive effect of the menthol anesthetic property. Both test and control sides had anesthesia that lasted similar times. The test side appeared to have more soreness than the control. After evaluating the formula, it appears that the solvent, which was alcohol in this batch, may be causing this sensation at the site post administration.

### B: Lidocaine Injectable: Ethanol Solvent 3%

A lidocaine preparation was prepared as in Example 5 having the following ingredient concentrations:

Lidocaine 1.9%

Epinephrine 1:900000

Menthol 0.4%

In this formulation the alcohol content was lowered from 10% by weight to 3% by weight to try to eliminate any soreness post administration. subjects were first administered 20 IUs of a commercially available 2% lidocaine with 1:100000 epinephrine product orally with the composition placed directly onto dorsal of tongue and the subjects asked to evaluate taste. Table 8 shows the results:

TABLE 8

| Test Subject | Rating |
|---|---|
| 1 | 7 |
| 2 | 8 |
| 3 | 8 |
| 4 | 7 |

After subjects had water and a small white plain cracker, then 30 minutes later, all 4 subjects were administered 20 IUs of the composition of this example orally with the composition placed directly onto dorsal of tongue and the subjects asked to evaluate taste. Table 9 shows the taste results.

TABLE 9

| Test Subject | Rating |
|---|---|
| 1 | 3 |
| 2 | 3 |
| 3 | 3 |
| 4 | 3 |

After taste test was performed anesthesia was administered via a block of the mental nerve using a split mouth design in which on one side of the patients mouth 100 IUs of 2% lido w/1:100 k epi was administered via mental block. At the same time 100 IUs of the composition of this example was administered on the contralateral side.

All subjects had profound anesthesia on both sides, with the test sites having slightly more then the control. This could be due to the additive effect of the menthol anesthetic property. Both test and control sides also had anesthesia that lasted similar times. By lowering the alcohol content, the soreness at the test site was decreased but still lingered slightly.

### Test C: Lidocaine Injectable: PEG Solvent

A lidocaine preparation was prepared as in Example 3 having the following ingredient concentrations:

Lidocaine 1%

Epinephrine 1:200000

Menthol 40 mg per cc

PEG used as solvent instead of alcohol

4 subjects were first administered 20 IUs of a commercially available 2% lidocaine with 1:100000 epinephrine productorally with the composition placed directly onto dorsal of tongue. Table 10 shows the results:

TABLE 10

| Test Subject | Rating |
|---|---|
| 1 | 7 |
| 2 | 8 |
| 3 | 8 |
| 4 | 7 |

After subjects had water and a small white plain cracker, then 10 minutes later, all 4 subjects were administered 20 IUs of the composition of this example orally with the composition placed directly onto dorsal of tongue and the subjects asked to evaluate taste. Table 11 shows the taste results.

TABLE 11

| Test Subject | Rating |
|---|---|
| 1 | 3 |
| 2 | 3 |
| 3 | 3 |
| 4 | 2 |

After taste test was performed anesthesia was administered via a block of the mental nerve using a split mouth design in which on one side of the patients mouth 100 IUs of 2% lido w/1:100 k epi was administered via mental block. At the same time 100 IUs of the composition of this example was administered on the contralateral side.

All subjects had profound anesthesia on both sides, with the test sites having slightly more then the control. This could be due to the additive effect of the menthol anesthetic property. Both test and control sides also had anesthesia that lasted similar times. By changing the solvent, soreness was eliminated and taste was altered to be pleasant instead of a bitter/metallic taste.

Test D: Lidocaine Injectable: PEG Solvent [what %/]

A lidocaine preparation was prepared as in Example 3 having the following ingredient concentrations:

Lidocaine 1%

Epinephrine 1:200000

Menthol 60 mg per cc

PEG used as solvent instead of alcohol

4 subjects were first administered 20 IUs of a commercially available 2% lidocaine with 1:100000 epinephrine product orally with the composition placed directly onto dorsal of tongue and the subjects asked to evaluate taste. Table 12 shows the results:

TABLE 12

| Test Subject | Rating |
|---|---|
| 1 | 7 |
| 2 | 8 |
| 3 | 8 |
| 4 | 7 |

After subjects had water and a small white plain cracker, then 10 minutes later, all 4 subjects were administered 20 IUs of the composition of this example orally with the composition placed directly onto dorsal of tongue and the subjects asked to evaluate taste. Table 13 shows the taste results.

TABLE 13

| Test Subject | Rating |
|---|---|
| 1 | 2 |
| 2 | 2 |
| 3 | 3 |
| 4 | 2 |

After taste test was performed anesthesia was administered via a block of the mental nerve using a split mouth design in which on one side of the patients mouth 100 IUs of 2% lido w/1:100 k epi was administered via mental block. At the same time 100 IUs of the composition of this example was administered on the contralateral side.

All subjects had profound anesthesia on both sides, with the test sites having slightly more then the control. This could be due to the additive effect of the menthol anesthetic property. Both test and control sides also had anesthesia that lasted similar times. By changing the solvent, soreness was greatly decreased (or should we say eliminated) and taste was altered to be pleasant instead of a bitter/metallic taste.

As can be seen, the compositions of the present invention clearly taste better to taste subjects.

Although the invention has been shown and described with respect to certain embodiments, it is obvious that equivalent alterations and modifications will occur to others skilled in the art upon the reading and understanding of this specification. In particular with regard to the various functions performed by the above described components, the terms (including any reference to a “means”) used to describe such components are intended to correspond, unless otherwise indicated, to any component which performs the specified function of the described component (e.g., that is functionally equivalent), even though not structurally equivalent to the disclosed structure which performs the function in the herein illustrated exemplary embodiments of the invention. In addition, while a particular feature of the invention may have been disclosed with respect to only one of several embodiments, such feature may be combined with one or more other features of the other embodiments as may be desired and advantageous for any given or particular application.

One of skill in the art will appreciate that substitutions and deviations from the above formulations may be permissible without departing from the spirit of the invention.

### REFERENCES

All references cited herein are incorporated by reference in their entirety.
1: Bonaparte J P, Corsten M, Rourke R. Reducing the flavour of oral lidocaine: randomized controlled trial assessing the efficacy of mint-flavoured mouthwash. J Otolaryngeal Head Neck Surgery 2010 February; 39 (1): 96-101
2: WHO Food Additives Series 10. 21-29 Apr. 1976. http://www.inchem.org/documents/jecfa/jecmono/v10je07.htm
3: Water-soluble menthol B hydroxypropyl cyclodextrin developed long-acting injection painkiller. Oct. 27, 2010. Can be found on http://www.cn-articles.com
4: (L)-Menthol-National Library of Medicine HSBD Database
5: Vibeke S. M. Bemson, Bertil Pettersson. The toxicity of menthol in short-term bioassays. Chemico-Biological Interactions Volume 46, Issue 2, September 1983 Pages 233-248.

We claim:

1. An injectable composition comprising an active agent,a flavor masking agent and a solvent.

2. The injectable composition of claim 1 wherein the active agent is selected from the group consisting of: lidocaine, novocaine, procaine, articaine, septocaine, marcainemidazolam, ketamine, propofol, fentanyl, bupivacaine, and mepivacaine; and where the flavoring masking agent is selected from ascorbic acid, menthol, peppermint, spearmint, wintergreen, butterscotch, chocolate, cocoa, pineapple, orange, any citrus based flavor, any mint based flavor, or a cooling agent.

3. The injectable composition of claim 2 wherein the active agent is lidocaine, bupivacaine, or mepivacaine, articaine and the cooling agent is menthol or a menthol derivative.

4. The injectable composition of claim 2 further comprising epinephrine.

5. The injectable composition of claim 3 wherein the active agent is lidocaine and the cooling agent is menthol or a menthol derivative.

6. The injectable composition of claim 3 wherein the active agent is bupivacaine and the cooling agent is menthol or a menthol derivative.

7. The injectable composition of claim 6 further comprising epinephrine.

8. The injectable composition of claim 3 wherein the active agent is mepivacaine and the cooling agent is menthol or a menthol derivative.

9. A method for masking the taste of an injectable composition comprising the administration of the composition of claim 1.

10. A method of blocking taste sensation in an injectable composition comprising the addition of a taste masking agent to an injectable composition having an active agent and injecting the tasked blocking composition to a patient.

11. The method of claim 10 where in the taste masking agent is ascorbic acid, menthol, peppermint, spearmint, wintergreen, butterscotch, chocolate, cocoa, pineapple, orange, any citrus based flavor, any mint based flavor, or a cooling agent.

12. The method of claim 11 where in the active agent is selected from lidocaine,novocaine, procaine, articaine, septocaine, marcainemidazolam, ketamine, propofol, fentanyl, bupivacaine, and mepivacaine.

13. The method of claim 10 wherein the active agent is lidocaine, bupivacaine, or mepivacaine and the cooling agent is menthol or a menthol derivative.

14. The method of claim 13 wherein the composition further comprises epinephrine.

15. The method of claim 10 wherein the active agent is lidocaine and the cooling agent is menthol or a menthol derivative.

16. The method of claim 10 wherein the active agent is bupivacaine and the cooling agent is menthol or a menthol derivative.

17. The method of claim 10 wherein the composition further comprises epinephrine.

18. The method of claim 10 wherein the active agent is mepivacaine and the cooling agent is menthol or a menthol derivative.

19. The composition of claim 1 wherein the solvent is selected from PEG (Polyethylene Glycols), Chloroform, Ethanol, Carvone, Dimethylsulfoxide, Citral, Tauroglycolate, Eugenol, Sodium Lauryl Sulfate, Oleic acid, Tween, N Methyl 2 pyrrilidione, 2 pyrol, PVP (Polyvinylpyrrilidones), or any alcohol and water in an amount sufficient to solubilize the active agent and the flavor masking agent.

20. The composition of claim 19 wherein the solvent is PEG in a range of 5-10% by weight.
